(19) United States
(12) Patent Application Publication
(10) Pub. No.: US 2006/0160189 A1
(43) Pub. Date: Jul. 20, 2006

(54) High-temperature enzyme starch-to-sugar conversion

(75) Inventor: Neal A. Hammond, Woodland, CA (US)

Correspondence Address: Robert Charles Hill, 235 Montgomery Street #821, San Francisco, CA, 94104, US

(73) Assignee: California Natural Products

(21) Appl. No.: 11/039,136

(22) Filed: Jan. 19, 2005

(51) Int. Cl. C12P 19/02; C12N 9/32; C12N 1/21

(52) U.S. Cl. 435/105; 435/204; 435/252.31

(57) ABSTRACT

A starch-to-sugars conversion method comprises mixing rice flour with water, alpha-amylase enzyme, and calcium chloride into a slurry with 27% solids. The slurry is liquefied at about 223° F. Glucoamylase and beta-amylase enzymes are added and the slurry is incubated. The action of the three enzymes is terminated. The slurry is centrifuged to produce dextrose equivalents, glucose, maltose, and maltotriose. The whole processing time is under two hours.

### BACKGROUND OF THE PRESENT INVENTION

1. Field of the Present Invention

Embodiments of the present invention relate to using high temperature alpha-amylase enzymes for rapid liquefaction and pasteurization of feed starches. The complete conversion to dextrose equivalents, glucose, maltose, and maltotriose is finished at reduced temperature by adding beta-amylase and glucoamylase enzymes.

2. Description of Related Art

Corn, potato, rice, sorghum, wheat, cassava, and other starchy foods comprise the major foods in human and animal diets. Starch molecules are synthesized naturally in such plants as glucose polymers linked together by the alpha-1,4 and alpha-1,6 glucosidic bonds. The carbon and energy stored in starch are broken down in the human digestive system with the help of enzyme amylases. These first break down the polymer to smaller assimilable sugars, which are eventually converted to basic glucose units.

There are two different possible molecular linkages for starch molecules. Amylose is an unbranched, single chain polymer of 500-2000 glucose subunits with only alpha-1,4 glucosidic bonds. A branched glucose polymer called amylopectin includes alpha-1,6 glucosidic linkages. The degree of branching in amylopectin is about 1:25 glucose units in the unbranched segments.

Glucose is stored in animal cells by glycogen. It has one branching per twelve glucose units. The degree of branching and the side chain length vary from source to source, but in general the more the chains are branched, the more the starch is soluble.

Starch is generally insoluble in water at room temperature. When an aqueous suspension of starch is heated, the hydrogen bonds will weaken, water will be absorbed, and the starch granules will swell. Depending on the relative location of the molecular chain bonds under attack, the products of such digestive process are dextrin, maltotriose, maltose, and glucose, etc. Dextrins are shorter, broken starch segments that form as the result of the random hydrolysis of internal glucosidic bonds. A molecule of maltotriose is formed if the third bond from the end of a starch molecule is cleaved. A molecule of maltose is formed if the point of attack is the second bond. A molecule of glucose results if the bond being cleaved is the terminal one. The initial step in random depolymerization is the splitting of large chains into various smaller sized segments. The breakdown of large particles drastically reduces the viscosity of gelatinized starch solution, resulting in a process called liquefaction because of the thinning of the solution. Saccharification is the final stage of depolymerization, and produces mono-saccharides, di-saccharides, and tri-saccharides.

A wide variety of organisms, including humans, can digest starch. Alpha-amylase is widely synthesized in nature. Human saliva and pancreatic secretion contain a large amount of alpha-amylase for starch digestion. The bond attacked by alpha-amylases depends on the sources of the enzymes. Two major classes of alpha-amylases are commercially produced through microbial fermentation. Based on the points of attack in the glucose polymer chain, they can be classified into two categories, liquefying and saccharifying.

Alpha-amylase attacks only the alpha-1,4 bonds, and belongs to the liquefying category. The hydrolysis reaction catalyzed by this class of enzymes is usually carried out only to the extent that the starch is rendered soluble enough to allow easy removal from starch-sized fabrics in the textile industry. The paper industry also uses liquefying amylases on the starch used in paper coating where breakage into the smallest glucose subunits is actually undesirable.

Fungal alpha-amylase is saccharifying category and attacks the second linkage from the nonreducing terminals of the straight segment. Such results in the splitting off of two glucose units at a time to yield maltose, a disaccharide. The bond breakage is more extensive in saccharifying enzymes than in liquefying enzymes. The starch chains are broken into small bits and pieces.

The amyloglucosidase (glucoamylase) component of an amylase preparation selectively attacks the last bond on the nonreducing terminals. It can act on both the alpha-1,4 and the alpha-1,6 glucosidic linkages at a relative rate of 1:20, resulting in the splitting off of simple glucose units into the solution. Fungal amylase and amyloglucosidase may be used together to convert starch to simple sugars. The practical applications of this type of enzyme mixture include the production of corn syrup and the conversion of cereal mashes to sugars in brewing.

Most industrial enzymes work in rather mild conditions, e.g., not too cold/hot, and not too acidic/alkaline. Many industrial chemical processes use high pressures and/or temperatures to drive reactions. Enzymes and catalysts have been used as a way to reduce the extremes needed, and therefore the fuel costs and equipment expenses. Enzymes typically work best 20° C.-60° C. (68° F.-140° F.), and a few developed strains can still thrive and do their work beyond this range. Using enzymes can reduce process times, lower capital costs, use energy more efficiently, use less water, produce less effluent, and reduce the risk of microbial contamination.

The conversion of starches to sugars is an important commercial industry with products that include syrups and other sweeteners. Fermentation can then be used to convert the sugars to alcohol, and such can be used as a fuel.

Compared to embodiments of the present invention, conventional methods can take much longer times to convert starches to sugar. For example, the following four United States Patents represent conventional commercial practice.

U.S. Pat. No. 4,744,992, issued to Mitchell, et al., May 17, 1988, titled NUTRITIONAL RICE MILK PRODUCTION, comprises selection of whole grain rice, either white or brown rice, which is liquefied, preferably with alpha-amylase enzymes, and then treated with relatively high levels of a glucosidase enzyme and/or a beta-amylase enzyme in a saccharifying step. The total enzymatic reaction time in both the liquefaction and saccharification steps is limited to prevent development of undesirable off-flavors to yield a non-allergenic rice milk product having surprising milk-like texture and functionality, the rice milk product being characterized by the absence of a rice-like flavor and having a preferred composition defined as follows: Soluble Complex Carbohydrates—10 to 70% of solids; Maltose—0 to 70% of solids; Glucose—5 to 70% of solids; Ash or Minerals—0.1 to 0.6% of solids; Protein and Fat—1 to 3.5% of solids; Fiber—0.05 to 0.4% of solids. The rice milk product can also be converted to a dried product.

U.S. Pat. No. 4,756,912, issued to Mitchell, et al., Jul. 12, 1988, titled RICE SYRUP SWEETENER PRODUCTION, liquefies and treats whole grain rice with high levels of a glucosidase enzyme and/or a combination with beta-amylase enzyme in a saccharification step. Total enzymatic reaction time is limited to about four hours for both the liquefaction and saccharification steps combined to prevent the development of undesirable off-flavors. The product of the saccharification step is partially clarified to remove substantially all rice fiber, but not other nutritional values and then concentrated to produce a preferred rice syrup sweetener which is cloudy in character and has a solids composition defined as follows: Soluble Complex Carbohydrates—About 10 to 70% of solids; Maltose—About 0 to 70% of solids; Glucose—About 5 to 70% of solids; Ash or Minerals—About 0.1 to 0.6% of solids; Protein and Fat—About 1 to 3.5% of solids; The rice syrup sweetener of the invention can be dried to produce dried rice sweeteners.

U.S. Pat. No. 4,873,112, issued to Mitchell, et al., Oct. 10, 1989, titled FRUIT CONCENTRATE SWEETENER AND PROCESS OF MANUFACTURE, discloses how a sweetener is formed from a hydrolyzed starch having a dextrose equivalent of about 5 to 25 and a clear fruit concentrate of at least about 40% soluble solids and about 0% insoluble solids to have about 40 to 65% complex carbohydrates, about 35 to 55% simple sugars from the fruit origin and about 0 to 5% nutritional components. The sweetener may be partially or substantially completely deflavorized and may be dried up to about 96 to 99% soluble solids. Further preferred steps of the process facilitate both deflavorization and drying while also yielding a sweetness level generally similar to sucrose with only about 50% simple sugars, the remainder being nutritionally desirable complex carbohydrates. The sweetener may be included in a variety of sweetened food and beverage products. The sweetener of the invention consists essentially of about 40 to 65% complex carbohydrates, about 35 to 55% simple sugars of a fruit origin, about 0% insoluble solids, about 0 to 5% nutritional components and about 0 to 3% of a sweetness potentiator, balance essentially water.

U.S. Pat. No. 4,876,096, issued to Mitchell, et al., Oct. 24, 1989, titled RICE SYRUP SWEETENER, discloses how whole grain rice, either white or brown rice, is liquefied and treated with high levels of a glucosidase enzyme and/or a combination with beta-amylase enzyme in a saccharification step. Total enzymatic reaction time is limited to about four hours for both the liquefaction and saccharification steps combined to prevent the development of undesirable off-flavors. The product of the saccharification step is partially clarified to remove substantially all rice fiber, but not other nutritional values and then concentrated to produce a preferred rice syrup sweetener which is cloudy in character and has a solids composition defined as follows:

| | |
|---|---|
| Soluble Complex Carbohydrates | About 10 to 70% of solids; |
| Maltose | About 0 to 70% of solids; |
| Glucose | About 5 to 70% of solids; |
| Ash or Minerals | About 0.1 to 0.6% of solids; |
| Protein and Fat | About 1 to 3.5% of solids; |

Such rice syrup sweetener can be dried to produce dried rice sweeteners.

### SUMMARY OF THE PRESENT INVENTION

Briefly, a starch-to-sugars conversion method embodiment of the present invention comprises mixing rice flour with water, alpha-amylase enzyme, and calcium chloride into a slurry with 27% solids. The slurry is liquefied at about 223° F. Glucoamylase and beta-amylase enzymes are added and the slurry is incubated. The action of the three enzymes is terminated. The slurry is centrifuged to produce dextrose equivalents, glucose, maltose, and maltotriose. The whole processing time is under two hours.

An advantage of embodiments of the present invention is that a starch hydrolysis method is provided that is relatively very fast.

Another advantage of embodiments of the present invention is that the high temperatures involved will pasteurize the products.

The above and still further objects, features, and advantages of embodiments of the present invention will become apparent upon consideration of the following detailed description of specific embodiments thereof, especially when taken in conjunction with the accompanying drawings.

### BRIEF DESCRIPTION OF THE DRAWINGS

FIG. 1 is a flowchart diagram of a first starch-to-sugar conversion method embodiment of the present invention; and

FIG. 2 is a flowchart diagram of a second starch-to-sugar conversion method embodiment of the present invention.

### DETAILED DESCRIPTION OF THE PRESENT INVENTION

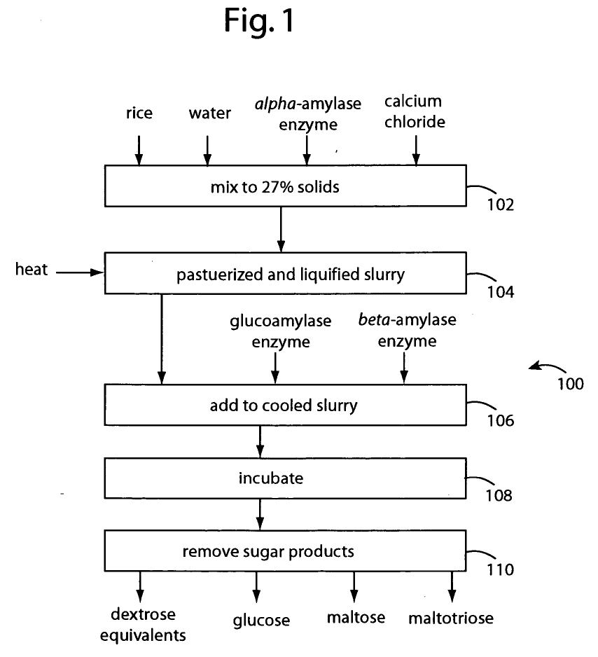

FIG. 1 illustrates a starch-to-sugar conversion embodiment of the present invention, referred to herein by the general reference numeral 100. As a general rule, enzyme activity doubles for every 10° C. rise in temperature. That is up until the point the high temperature denatures the enzyme. Conventional alpha-amylase has optimal enzyme activity around 155°-158° F. Novozymes' TERMAMYL is a liquid enzyme preparation of a heat-stable alpha-amylase produced by a strain of Bacillus licheniformis. (Novozymes A/S, Krogshoejvej 36, 2880 Bagsvaerd, Denmark.) TERMAMYL is marketed for use in the starch industry for the continuous liquefaction of starch at temperatures up to 105° C.-110° C. In the sugar industry, TERMAMYL is marketed to break down the starch present in cane juice.

Embodiments of the present invention therefore use high temperature enzymes to liquefy the starch quickly. The starch-to-sugar conversion method 100 comprises a step 102 for mixing together a water, a starchy food, and an alpha-amylase enzyme into a slurry. A step 104 is for heating the slurry to about 105° C. to both liquefy and pasteurize the mixture. A step 106 is used for adding both glucoamylase and beta-amylase enzymes to a liquefied slurry obtained in the step of heating. Novozymes' amyloglucosidase AMG has a pH optimum of about 4.0 and a temperature optimum of about 75° C. Beta-amylase has optimal enzyme activity around 140°-149°. It attacks chains of glucose only from the non-reducing end to form maltose, a simple sugar made up of two glucose molecules. A step 108 incubates the slurry for less than one hour and then terminates the enzyme reaction. A final step 110 removes the sugars converted from starches, e.g., for use in commercial products. For example, these sugars can include dextrose equivalents, glucose, maltose, and maltotriose.

In embodiments of the present invention, the starch-to-sugar conversion time is greatly reduced following thermoamylase treatment, because the conditions required by amyloglucosidase (glucoamylase) enzymes are optimized. Amyloglucosidase can be used to convert non-fermentable sugars into fermentable ones.

Experimental starting solutions of starches were prepared by adding enough water to 33-grams of material to yield 100-grams total weight. The high temperature alpha-amylase was added, and the mixture heated to 105° C. for six minutes. Then reduced to 95° C. for 20 minutes. When liquefaction was complete, the temperature was reduced to 60° C. and amyloglucosidase (AMG 150, liquid, Novo Industrials) was added.

The optimal amount of amyloglucosidase was one percent volume/weight achieved a complete starch-to-sugar conversion in less than two hours. The resulting products can be used directly as sugar syrup, or fermentation substrates to produce single-cell protein and alcohol for fuels.

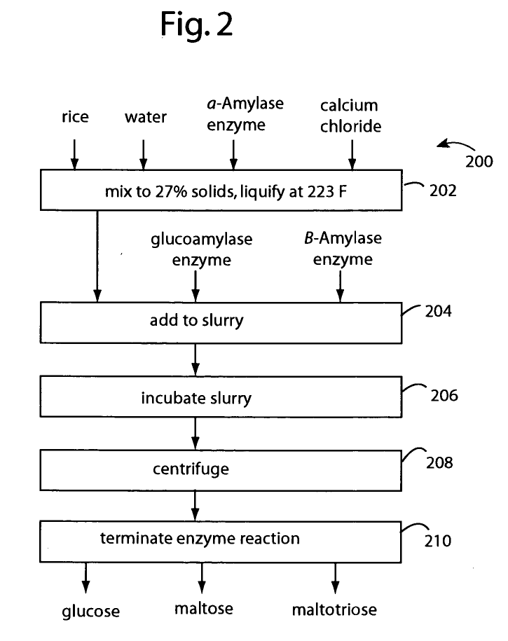

FIG. 2 illustrates another starch-to-sugar conversion embodiment of the present invention, referred to herein by the general reference numeral 200. The method 200 mixes rice, as a source of starch, with water alpha-amylase, and calcium chloride. The starch is liquefied in a step 202. In a step 204, glucoamylase and beta-amylase enzymes are added. These mixture is incubated in a step 206. The solids are dried out using a centrifuge in a step 208. The enzyme reactions are terminated in a step 210 by cooling.

Enzyme technology applications in rural communities must use simple methods to be successful. High-technology whole cell systems could be used to develop desired cell lines. The micro-organisms themselves can provide the technology transfer mechanisms. Genetic engineering, mixed culture development, and microbial selection can be used to create self-replicating micro-organisms in the rural community. Hydrolytic processes will must probably provide the first immediate impact in world development. There is a wide variety of natural polymers whose hydrolytic degradation could be used to improve existing raw materials or provide new sources.

In experiments, 400-grams of rice flour slurry with 28% solids were subjected to three types of saccharification following high temperature alpha-amylase liquefaction. In a first example (Table I), 0.3588 grams of glucoamylase enzyme and 0.1788 grams of beta-amylase were used. In a second (2×) example (Table II), 0.7096 grams of glucoamylase enzyme and 0.356 grams of beta-amylase were used. In a third (3×) example (Table III), 1.0644 grams of glucoamylase enzyme and 0.5364 grams of beta-amylase were used. The liquefied material was mixed at 140° F. with the enzymes at different concentrations and times. The times varied from ten to fifty minutes.

Afterwards, the activity was stopped by chilling in an ice bath to 32° F. The result was centrifuged to remove the liquid and the solids were analyzed for glucose polymers.

TABLE I
First Example

| | minutes | | | | |
|---|---|---|---|---|---|
| products | 0 | 10 | 20 | 30 | 50 |
| dextrose equivalent | 22.0 | 47.0 | 56.0 | 63.0 | 75.0 |
| glucose | 4.4 | 27.8 | 40.1 | 50.8 | 53.3 |
| maltose | 11.6 | 21.5 | 28.6 | 23.1 | 40.3 |
| maltotriose | 14.2 | 16.8 | 0.6 | 0.7 | 1.2 |

Even at zero minutes of reaction, the combination of alpha-amylase, glucoamylase, and beta-amylase enzymes produced a significant hydrolysis of liquefied starch to glucose, maltose, and maltotriose. The results of such hydrolysis were as good as those for the conventional time of fifty minutes.

TABLE II
Second Example

| | minutes | | |
|---|---|---|---|
| products | 10 | 20 | 30 |
| dextrose equivalent | 60.0 | 70.0 | 82.0 |
| glucose | 43.9 | 59.9 | 68.9 |
| maltose | 29.1 | 18.2 | 10.1 |
| maltotriose | 1.6 | 0.7 | 0.7 |

The combination of alpha-amylase, glucoamylase, and beta-amylase enzymes produced a significant hydrolysis of liquefied starch to glucose, maltose, and maltotriose. The results of such hydrolysis were as good as those for the conventional time of fifty minutes.

TABLE III
Third Example

| | minutes | | |
|---|---|---|---|
| products | 10 | 20 | 30 |
| dextrose equivalent | 68.0 | 67.0 | 82.0 |
| glucose | 56.4 | 63.9 | 80.5 |
| maltose | 19.8 | 5.7 | 1.4 |
| maltotriose | 2.8 | 0.8 | 0.6 |

The combination of alpha-amylase, glucoamylase, and beta-amylase enzymes produced a significant hydrolysis of liquefied starch to glucose, maltose, and maltotriose. The results of such hydrolysis were as good as those for the conventional time of fifty minutes.

Although particular embodiments of the present invention have been described and illustrated, such is not intended to limit the present invention. Modifications and changes will no doubt become apparent to those skilled in the art, and it is intended that the present invention only be limited by the scope of the appended claims.

The present invention claimed is:

1. Starch-to-sugar conversion method, comprising:
mixing together a water, a starchy food, and an alpha-amylase enzyme into a slurry;
heating said slurry to about 105° C. to both liquefy and pasteurize the mixture;
adding both glucoamylase and beta-amylase enzymes to a liquefied slurry obtained in the step of heating;
incubating said slurry for less than one hour and then terminating the enzyme reaction; and
removing sugars converted from starches for use in commercial products.

2. The conversion method of claim 1, wherein:
the step of mixing includes using rice flour as said starchy food and said water is adjusted to result in a slurry with about 27% solids.

3. The conversion method of claim 1, wherein:
the step of mixing includes using a heat-stable alpha-amylase produced by a genetically-modified strain of Bacillus licheniformis, wherein said heat stability of the enzyme is not related to any changes made by genetic modification and the gene transfer is simply to enhance production of the enzyme.
